(19) United States
(12) Patent Application Publication
(10) Pub. No.: US 2015/0126626 A1
(43) Pub. Date: May 7, 2015

(54) LIQUID CATALYST FOR METHANATION OF CARBON DIOXIDE

(71) Applicant: Wuhan Kaidi Engineering Technology Research Institute Co., Ltd., Wuhan (CN)

(72) Inventors: Yanfeng ZHANG, Wuhan (CN); Xiaodong ZHAN, Wuhan (CN); Xingcai ZHENG, Wuhan (CN); Zhilong WANG, Wuhan (CN); Zhangjian FANG, Wuhan (CN); Yongjie XUE, Wuhan (CN); Leiming TAO, Wuhan (CN)

(21) Appl. No.: 14/569,769

(22) Filed: Dec. 14, 2014

Related U.S. Application Data
(63) Continuation-in-part of application No. PCT/CN2013/076858, filed on Jun. 6, 2013.

(30) Foreign Application Priority Data
Jun. 15, 2012 (CN) 201210196937.1

(51) Int. Cl. B01J 31/06; B01J 23/89; C07C 1/12; B01J 23/78; B01J 23/883; B01J 31/02; B01J 23/83

(52) U.S. Cl. CPC B01J 31/06; B01J 31/0285; B01J 23/892; B01J 23/83; B01J 23/78; B01J 23/883; C07C 1/12; B01J 2531/847; B01J 2231/625
USPC 518/700; 502/173; 502/159; 502/167

(57) ABSTRACT

A liquid catalyst for methanation of carbon dioxide, including an amphiphilic ionic liquid and a metal active component dispersed in the amphiphilic ionic liquid. The metal active component is dispersed in the amphiphilic ionic liquid in the form of stable colloid. The colloid is spherical and has a particle size of between 0.5 and 20 nm. The metal active component includes a first metal active component and a second metal active component. The first metal active component includes nickel. The second metal active component is selected from the group consisting of lanthanum, cerium, molybdenum, ruthenium, ytterbium, rhodium, palladium, platinum, potassium, magnesium, or a mixture thereof. The molar ratio of the first metal active component to the second metal active component is between 10:0.1 and 10:2.

### CROSS-REFERENCE TO RELATED APPLICATIONS

This application is a continuation-in-part of International Patent Application No. PCT/CN2013/076858 with an international filing date of Jun. 6, 2013, designating the United States, now pending, and further claims priority benefits to Chinese Patent Application No. 201210196937.1 filed Jun. 15, 2012. The contents of all of the aforementioned applications, including any intervening amendments thereto, are incorporated herein by reference. Inquiries from the public to applicants or assignees concerning this document or the related applications should be directed to: Matthias Scholl P. C., Attn.: Dr. Matthias Scholl Esq., 245 First Street, 18th Floor, Cambridge, Mass. 02142.

### BACKGROUND OF THE INVENTION

1. Field of the Invention

The invention relates to a liquid catalyst for methanation of carbon dioxide.

2. Description of the Related Art

Conventional preparation methods of a nickel-based catalyst for methanation of CO₂ consume large energy, and the activity of the catalyst prepared by the conventional methods decreases greatly under high temperature and high water vapor pressure. In addition, the catalyst layer deposited on the surface of the substrate tends to erode and detach from the surface, which greatly limits the application of the CO₂ methanation catalyst.

### SUMMARY OF THE INVENTION

In view of the above-described problems, it is one objective of the invention to provide a liquid catalyst for methanation of carbon dioxide under liquid phase and low temperature conditions.

To achieve the above objective, in accordance with one embodiment of the invention, there is provided a liquid catalyst for methanation of carbon dioxide, comprising an amphiphilic ionic liquid and a metal active component dispersed in the amphiphilic ionic liquid. The metal active component is dispersed in the amphiphilic ionic liquid in the form of stable colloid, and the colloid is spherical and has a particle size of between 0.5 and 20 nm. The metal active component comprises a first metal active component and a second metal active component. The first metal active component comprises nickel, and the second metal active component is selected from the group consisting of lanthanum, cerium, molybdenum, ruthenium, ytterbium, rhodium, palladium, platinum, potassium, magnesium, or a mixture thereof. A molar ratio of the first metal active component to the second metal active component is between 10:0.1 and 10:2.

In a class of this embodiment, a molar ratio of the amphiphilic ionic liquid to the first metal active component is between 10:0.1 and 10:2.

In a class of this embodiment, the amphiphilic ionic liquid is an amphiphilic small molecular ionic liquid or an amphiphilic polymer ionic liquid; the amphiphilic small molecular ionic liquid comprises N-alkyl pyridinium hydrochloride, N-alkyl pyridinium tetrafluoroborate, 1,3-dialkyl-imidazole tetrafluoroborate, 1,3-dialkyl-imidazole hydrochloride, 1,3-dialkyl-imidazole hexafluorophosphate, and the alkyl has a carbon chain length of between 8 and 18; the amphiphilic polymer ionic liquid comprises poly-1-(4-styryl)-3-methylimidazole hexafluorophosphate, poly-1-(4-styryl)-3-methylimidazole (trifluoromethyl)sulfonyl imide, poly-1-(4-styryl)-3-methylimidazole tetrafluoroborate, poly-1-(4-styryl)-3-butyllimidazole tetrafluoroborate, poly-1-(4-styryl)-3-butyllimidazole polypyrrolidone hydrochloride.

In accordance with another embodiment of the invention, there provided is a method for preparation of a liquid catalyst for methanation of carbon dioxide, the method comprising:
1) dispersing a soluble salt of a metal active component and an amphiphilic ionic liquid in a liquid medium to yield a mixed solution, the metal active component comprising a first metal active component and a second metal active component with a molar ratio of metal ions thereof being between 10:0.1 and 10:2;
2) regulating a pH value of the mixed solution to 8-10, stirring for between 1 and 3 hours, charging hydrogen to reduce the mixed solution at a temperature of between 100 and 200° C. and a pressure of between 0.1 and 4.0 MPa for between 1 and 6 hours; and
3) centrifuging, filtering, washing, and drying a resulting product.

In a class of this embodiment, the soluble salt of the first metal active component is a nitrate, acetate, or chloride of nickel, and the soluble salt of the second metal active component is a nitrate thereof.

In a class of this embodiment, a molar ratio of the amphiphilic ionic liquid to metal ions of the soluble salt of the first metal active component is between 10:0.1 and 10:2, and a total concentration of the metal ions in the mixed solution is between 0.0001 and 0.01 mol/L.

In a class of this embodiment, the liquid medium comprises water, alcohols, tetrahydrofuran, acetonitrile, 1,4-dioxane, n-hexane, and cyclohexane.

The invention further provides a method for methanation of carbon dioxide, the method comprising adding to a reactor the liquid catalyst and a solvent, controlling a concentration of the liquid catalyst in a reaction system to be between 0.0001 and 0.01 mol/L, charging hydrogen and carbon dioxide to the reactor, heating the reactor to be between 100 and 120° C., and allowing the hydrogen and the carbon dioxide to react at a pressure of between 0.1 and 4.0 MPa for between 1 and 6 hours.

In a class of this embodiment, the solvent comprises water, alcohols, tetrahydrofuran, acetonitrile, 1,4-dioxane, n-hexane, and cyclohexane.

In a class of this embodiment, a molar ratio of the hydrogen to the carbon dioxide is between 0.5:1 and 5:1, preferably, 4.0. The catalytic pressure is preferably at 3 MPa.

CO₂ methanation reaction releases strong heat energy and low temperature can accelerate the reaction. Based on the theory and taking into account a unique structure and confinement effect of a metal nanoparticle agent, the invention provides a liquid catalyst for methanation of CO₂ under liquid phase and low temperature conditions. The catalytic activity, methane selectivity and stability of the catalyst are all controllable.

Advantages according to embodiments of the invention are summarized as follows:

1. The metal active component of the CO₂ methanation catalyst has small particle size (0.5-20 nm) and narrow particle size distribution, and is uniformly dispersed in the amphiphilic ionic liquid to form stable collide. No coagulation occurs before reaction. After reaction, the catalyst is easy to separate from the products such as methane and reaction mediums, which is beneficial to the recycling of the catalyst.

2. The amphiphilic ionic liquid can prevent the coagulation of the metal active nanoparticles, facilitate CO₂ to adsorb on the surface of the metal active nanoparticles, enhance the CO₂ concentration in the active site, and facilitate the CO₂ methanation. The CO₂ methanation can be achieved at liquid state and 100-200° C. The catalyst has good low temperature catalytic activity, methane selectivity, and good thermal stability. The common problems such as falling off of solid catalysts at high temperature and the coagulation of the active components are avoided. The application of the catalyst can save the energy consumption and reduce the production costs.

3. The preparation method of the catalyst is simple, has low costs, and is suitable for popularization.

### BRIEF DESCRIPTION OF THE DRAWINGS

The invention is described hereinbelow with reference to the accompanying drawings, in which:

FIG. 1 is a SEM of a liquid catalyst for methanation of carbon dioxide prepared in Example 1; and

FIG. 2 is a diagram showing the activity of a liquid catalyst for methanation of carbon dioxide after 300 hours' methanation reaction.

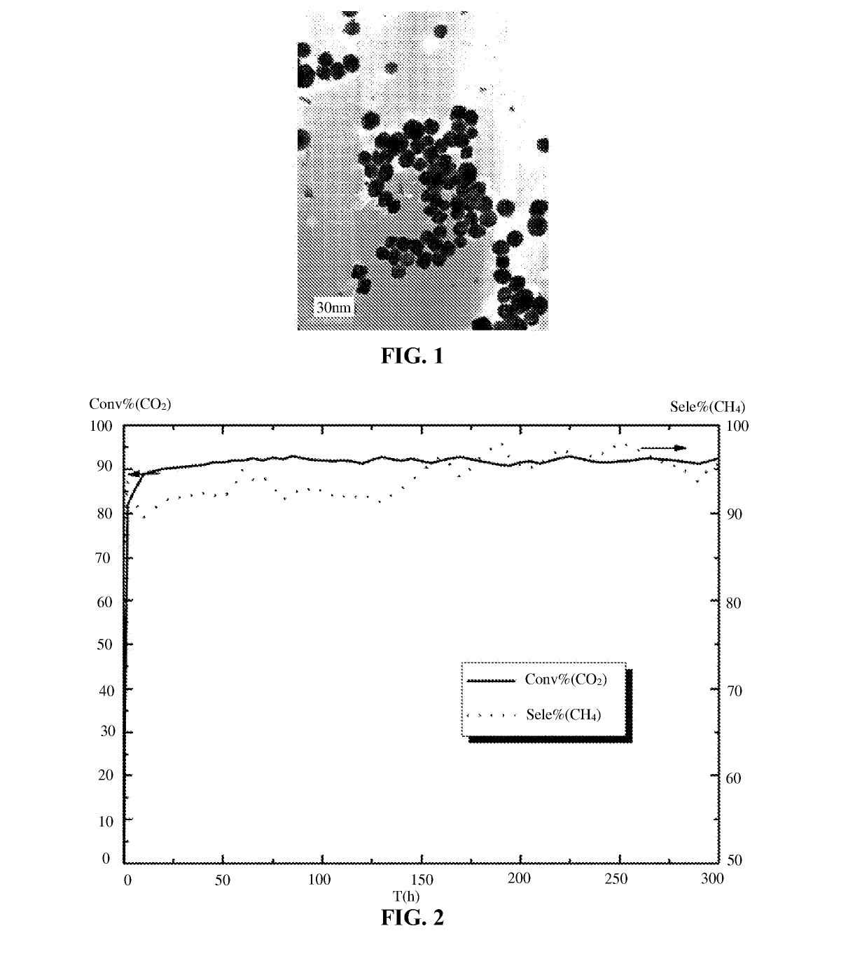

### DETAILED DESCRIPTION OF THE EMBODIMENTS

For further illustrating the invention, experiments detailing a liquid catalyst for methanation of carbon dioxide are described below. It should be noted that the following examples are intended to describe and not to limit the invention.

### Example 1

Nickel nitrate, magnesium nitrate, and an ionic liquid of 1-hexadecyl-3-methylimidazole hydrochloride were mixed according to a molar ratio thereof of 1:0.2:20 and dispersed in a liquid medium, for example, water, to yield a mixed solution. In the mixed solution, the molar concentration of nickel nitrate was 2.79×10⁻⁴ mol/L. The pH value of the mixed solution was adjusted using aqueous ammonia to 9.0. The mixed solution was stirred for 3 hours and transferred to a reactor. The reactor was heated to 150° C. and aerated with hydrogen at the pressure of 3.0 MPa for 2 hours. The resulting product was centrifuged, filtered, washed respectively with water and alcohol, and dried. TEM measurement showed that, the nanoparticles of the metal active component of the liquid catalyst for methanation of carbon dioxide had an average particle size of 11.8 nm, with a narrow and uniform distribution.

The liquid catalyst is applied to methanation of CO₂. Specifically, the liquid catalyst and water were added to a reactor. The concentration of the liquid catalyst in the reaction system was controlled at 0.005 mol/L. CO₂ and H₂ were charged to the reactor according to the equation nH₂: nCO₂=4, heated to 150° C., and allowed to react for 2 hours at the pressure of 3 MPa. The CO₂ conversion rate and methane selectivity were listed in Table 1.

TABLE 1
CO₂ conversion rate and methane selectivity

| Example | Conv. % (C0₂) | Sele. % (CH₄) |
|---|---|---|
| 1 | 88.3 | 93.5 |
| 2 | 80.6 | 92.5 |
| 3 | 86.7 | 89.3 |
| 4 | 73.2 | 91.2 |
| 5 | 85.2 | 81.3 |
| 6 | 75.9 | 84.6 |
| 7 | 62.8 | 89.5 |
| 8 | 65.7 | 75.3 |
| 9 | 80.6 | 86.9 |
| 10 | 59.3 | 93.1 |

### Example 2

Nickel acetate, lanthanum nitrate, and an ionic liquid of 1-octadecyl-3-methylimidazole hexafluorophosphate were mixed according to a molar ratio thereof of 1:0.15:20 and dispersed in a liquid medium, for example, alcohol, to yield a mixed solution. In the mixed solution, the molar concentration of nickel acetate was 3.61×10⁻⁴ mol/L. The pH value of the mixed solution was adjusted using triethylamine to 8.0. The mixed solution was stirred for 2 hours and transferred to a reactor. The reactor was heated to 100° C. and aerated with hydrogen at the pressure of 3.0 MPa for 1.5 hours. The resulting product was centrifuged, filtered, washed respectively with water and alcohol, and dried.

The liquid catalyst is applied to methanation of CO₂. Specifically, the liquid catalyst and water were added to a reactor. The concentration of the liquid catalyst in the reaction system was controlled at 0.005 mol/L. CO₂ and H₂ were charged to the reactor according to the equation nH₂: nCO₂=4, heated to 150° C., and allowed to react for 2 hours at the pressure of 3 MPa. The CO₂ conversion rate and methane selectivity were listed in Table 1.

### Example 3

Nickel nitrate, cerium nitrate, and an ionic liquid of N-dodecyl pyridinium tetrafluoroborate were mixed according to a molar ratio thereof of 1:0.15:18 and dispersed in a liquid medium, for example, acetonitrile, to yield a mixed solution. In the mixed solution, the molar concentration of nickel nitrate was 1.32×10⁻³ mol/L. The pH value of the mixed solution was adjusted using diisopropyl tert-butylamine to 10.0. The mixed solution was stirred for 3 hours and transferred to a reactor. The reactor was heated to 150° C. and aerated with hydrogen at the pressure of 3.0 MPa for 1.5 hours. The resulting product was centrifuged, filtered, washed, and dried.

The liquid catalyst is applied to methanation of CO₂. Specifically, the liquid catalyst and water were added to a reactor. The concentration of the liquid catalyst in the reaction system was controlled at 0.005 mol/L. CO₂ and H₂ were charged to the reactor according to the equation nH₂: nCO₂=4, heated to 120° C., and allowed to react for 3 hours at the pressure of 1 MPa. The CO₂ conversion rate and methane selectivity were listed in Table 1.

### Example 4

Nickel nitrate, molybdenum nitrate, and an ionic liquid of poly-1-(4-styryl)-3-methylimidazole (trifluoromethyl)sulfonyl imide were mixed according to a molar ratio thereof of 1:0.12:16 and dispersed in a liquid medium, for example, water, to yield a mixed solution. In the mixed solution, the molar concentration of nickel nitrate was 6.35×10⁻⁴ mol/L. The pH value of the mixed solution was adjusted using aqueous ammonia to 10.0. The mixed solution was stirred for 2.5 hours and transferred to a reactor. The reactor was heated to 200° C. and aerated with hydrogen at the pressure of 2.0 MPa for 2 hours. The resulting product was centrifuged, filtered, washed, and dried.

The liquid catalyst is applied to methanation of CO₂. Specifically, the liquid catalyst and dioxane were added to a reactor. The concentration of the liquid catalyst in the reaction system was controlled at 0.005 mol/L. CO₂ and H₂ were charged to the reactor according to the equation nH₂: nCO₂=4, heated to 150° C., and allowed to react for one hour at the pressure of 13 MPa. The CO₂ conversion rate and methane selectivity were listed in Table 1.

### Example 5

Nickel nitrate, ruthenium nitrate, potassium nitrate, and an ionic liquid of N-hexadecyl pyridinium tetrafluoroborate were mixed according to a molar ratio thereof of 1:0.1:0.1:20 and dispersed in a liquid medium, for example, tetrahydrofuran, to yield a mixed solution. In the mixed solution, the molar concentration of nickel nitrate was 8.1×10⁻⁴ mol/L. The pH value of the mixed solution was adjusted using aqueous ammonia to 8.0. The mixed solution was stirred for one hour and transferred to a reactor. The reactor was heated to 150° C. and aerated with hydrogen at the pressure of 1.0 MPa for 5 hours. The resulting product was centrifuged, filtered, washed, and dried.

The liquid catalyst is applied to methanation of CO₂. Specifically, the liquid catalyst and tetrahydrofuran were added to a reactor. The concentration of the liquid catalyst in the reaction system was controlled at 0.005 mol/L. CO₂ and H₂ were charged to the reactor according to the equation nH₂: nCO₂=4, heated to 110° C., and allowed to react for 2.5 hours at the pressure of 3 MPa. The CO₂ conversion rate and methane selectivity were listed in Table 1.

### Example 6

Nickel nitrate, ytterbium nitrate, and an ionic liquid of poly-1-(4-styryl)-3-methylimidazole tetrafluoroborate were mixed according to a molar ratio thereof of 1:0.15:15 and dispersed in a liquid medium, for example, cyclohexane, to yield a mixed solution. In the mixed solution, the molar concentration of nickel nitrate was 9.81×10⁻⁴ mol/L. The pH value of the mixed solution was adjusted using aqueous ammonia to 8.0. The mixed solution was stirred for 2 hours and transferred to a reactor. The reactor was heated to 150° C. and aerated with hydrogen at the pressure of 0.1 MPa for 3 hours. The resulting product was centrifuged, filtered, washed, and dried.

The liquid catalyst is applied to methanation of CO₂. Specifically, the liquid catalyst and cyclohexane were added to a reactor. The concentration of the liquid catalyst in the reaction system was controlled at 0.005 mol/L. CO₂ and H₂ were charged to the reactor according to the equation nH₂: nCO₂=0.5, heated to 190° C., and allowed to react for 1.5 hours at the pressure of 4 MPa. The CO₂ conversion rate and methane selectivity were listed in Table 1.

### Example 7

Nickel nitrate, rhodium nitrate, and an ionic liquid of poly-1-(4-styryl)-3-butyllimidazole tetrafluoroborate were mixed according to a molar ratio thereof of 1:0.16:10 and dispersed in a liquid medium, for example, alcohol, to yield a mixed solution. In the mixed solution, the molar concentration of nickel nitrate was 1.28×10⁻³ mol/L. The pH value of the mixed solution was adjusted using aqueous ammonia to 8.0. The mixed solution was stirred for 3 hours and transferred to a reactor. The reactor was heated to 100° C. and aerated with hydrogen at the pressure of 0.5 MPa for 2 hours. The resulting product was centrifuged, filtered, washed, and dried.

The liquid catalyst is applied to methanation of CO₂. Specifically, the liquid catalyst and alcohol were added to a reactor. The concentration of the liquid catalyst in the reaction system was controlled at 0.003 mol/L. CO₂ and H₂ were charged to the reactor according to the equation nH₂: nCO₂=4, heated to 110° C., and allowed to react for 2 hours at the pressure of 3 MPa. The CO₂ conversion rate and methane selectivity were listed in Table 1.

### Example 8

Nickel nitrate, palladium nitrate, and an ionic liquid of N-octyl pyridinium tetrafluoroborate were mixed according to a molar ratio thereof of 1:0.01:100 and dispersed in a liquid medium, for example, dioxane, to yield a mixed solution. In the mixed solution, the molar concentration of nickel nitrate was 3.18×10⁻³ mol/L. The pH value of the mixed solution was adjusted using aqueous ammonia to 8.0. The mixed solution was stirred for 3 hours and transferred to a reactor. The reactor was heated to 200° C. and aerated with hydrogen at the pressure of 3.0 MPa for 2 hours. The resulting product was centrifuged, filtered, washed, and dried.

The liquid catalyst is applied to methanation of CO₂. Specifically, the liquid catalyst and dioxane were added to a reactor. The concentration of the liquid catalyst in the reaction system was controlled at 0.0005 mol/L. CO₂ and H₂ were charged to the reactor according to the equation nH₂: nCO₂=3, heated to 120° C., and allowed to react for 2 hours at the pressure of 3 MPa. The CO₂ conversion rate and methane selectivity were listed in Table 1.

The catalytic reaction was carried out for consecutive 300 hours, and the CO₂ conversion rate and methane selectivity were measured along with the reaction time. As shown in FIG. 2, the liquid catalyst for methanation of carbon dioxide had good stability.

### Example 9

Nickel nitrate, platinum nitrate, and an ionic liquid of poly-1-(4-styryl)-3-butyllimidazole polypyrrolidone hydrochloride were mixed according to a molar ratio thereof of 1:0.05:5 and dispersed in a liquid medium, for example, n-hexane, to yield a mixed solution. In the mixed solution, the molar concentration of nickel nitrate was 6.28×10⁻⁴ mol/L. The pH value of the mixed solution was adjusted using aqueous ammonia to 8.0. The mixed solution was stirred for 3 hours and transferred to a reactor. The reactor was heated to 100° C. and aerated with hydrogen at the pressure of 4.0 MPa for 2 hours. The resulting product was centrifuged, filtered, washed, and dried.

The liquid catalyst is applied to methanation of CO₂. Specifically, the liquid catalyst and water were added to a reactor. The concentration of the liquid catalyst in the reaction system was controlled at 0.00015 mol/L. CO₂ and H₂ were charged to the reactor according to the equation nH₂: nCO₂=5, heated to 200° C., and allowed to react for 2 hours at the pressure of 2 MPa. The CO₂ conversion rate and methane selectivity were listed in Table 1.

### Example 10

Nickel nitrate, cerium nitrate, magnesium nitrate, and an ionic liquid of 1,3-dioctyl-imidazole tetrafluoroborate were mixed according to a molar ratio thereof of 1:0.15:0.05:10 and dispersed in a liquid medium, for example, acetonitrile, to yield a mixed solution. In the mixed solution, the molar concentration of nickel nitrate was 3.18×10⁻³ mol/L. The pH value of the mixed solution was adjusted using triethylamine to 9.0. The mixed solution was stirred for 3 hours and transferred to a reactor. The reactor was heated to 150° C. and aerated with hydrogen at the pressure of 3.0 MPa for 2 hours. The resulting product was centrifuged, filtered, washed, and dried.

The liquid catalyst is applied to methanation of CO₂. Specifically, the liquid catalyst and n-hexane were added to a reactor. The concentration of the liquid catalyst in the reaction system was controlled at 0.01 mol/L. CO₂ and H₂ were charged to the reactor according to the equation nH₂: nCO₂=4, heated to 100° C., and allowed to react for 2 hours at the pressure of 3 MPa. The CO₂ conversion rate and methane selectivity were listed in Table 1.

The CO₂ conversion rate and methane selectivity are calculated according to the following equations:

CO₂ conversion rate=(mole number of reacted CO₂/total mole number of CO₂ in raw gas)×100%

Methane selectivity=(mole number of produced methane/total mole number of reacted CO₂)×100%

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A liquid catalyst for methanation of carbon dioxide, comprising an amphiphilic ionic liquid and a metal active component dispersed in the amphiphilic ionic liquid, wherein
the metal active component is dispersed in the amphiphilic ionic liquid in the form of stable colloid, and the colloid is spherical and has a particle size of between 0.5 and 20 nm;
the metal active component comprises a first metal active component and a second metal active component;
the first metal active component comprises nickel, and the second metal active component is selected from the group consisting of lanthanum, cerium, molybdenum, ruthenium, ytterbium, rhodium, palladium, platinum, potassium, magnesium, or a mixture thereof; and
a molar ratio of the first metal active component to the second metal active component is between 10:0.1 and 10:2.

2. The liquid catalyst of claim 1, wherein a molar ratio of the amphiphilic ionic liquid to the first metal active component is between 10:0.1 and 10:2.

3. The liquid catalyst of claim 1, wherein
the amphiphilic ionic liquid is an amphiphilic small molecular ionic liquid or an amphiphilic polymer ionic liquid;
the amphiphilic small molecular ionic liquid comprises N-alkyl pyridinium hydrochloride, N-alkyl pyridinium tetrafluoroborate, 1,3-dialkyl-imidazole tetrafluoroborate, 1,3-dialkyl-imidazole hydrochloride, 1,3-dialkyl-imidazole hexafluorophosphate, and the alkyl has a carbon chain length of between 8 and 18;
the amphiphilic polymer ionic liquid comprises poly-1-(4-styryl)-3-methylimidazole hexafluorophosphate, poly-1-(4-styryl)-3-methylimidazole (trifluoromethyl)sulfonyl imide, poly-1-(4-styryl)-3-methylimidazole tetrafluoroborate, poly-1-(4-styryl)-3-butyllimidazole tetrafluoroborate, poly-1-(4-styryl)-3-butyllimidazole polypyrrolidone hydrochloride.

4. A method for preparation of a liquid catalyst for methanation of carbon dioxide, the method comprising:
1) dispersing a soluble salt of a metal active component and an amphiphilic ionic liquid in a liquid medium to yield a mixed solution, the metal active component comprising a first metal active component and a second metal active component with a molar ratio of metal ions thereof being between 10:0.1 and 10:2;
2) regulating a pH value of the mixed solution to 8-10, stirring for between 1 and 3 hours, charging hydrogen to reduce the mixed solution at a temperature of between 100 and 200° C. and a pressure of between 0.1 and 4.0 MPa for between 1 and 6 hours; and
3) centrifuging, filtering, washing, and drying a resulting product.

5. The method of claim 4, wherein the soluble salt of the first metal active component is a nitrate, acetate, or chloride of nickel, and the soluble salt of the second metal active component is a nitrate thereof.

6. The method of claim 4, wherein a molar ratio of the amphiphilic ionic liquid to metal ions of the soluble salt of the first metal active component is between 10:0.1 and 10:2, and a total concentration of the metal ions in the mixed solution is between 0.0001 and 0.01 mol/L.

7. The method of claim 4, wherein the liquid medium comprises water, alcohols, tetrahydrofuran, acetonitrile, 1,4-dioxane, n-hexane, and cyclohexane.

8. A method for methanation of carbon dioxide, the method comprising adding to a reactor the liquid catalyst of claim 1 and a solvent, controlling a concentration of the liquid catalyst in a reaction system to be between 0.0001 and 0.01 mol/L, charging hydrogen and carbon dioxide to the reactor, heating the reactor to be between 100 and 120° C., and allowing the hydrogen and the carbon dioxide to react at a pressure of between 0.1 and 4.0 MPa for between 1 and 6 hours.

9. The method of claim 8, wherein the solvent comprises water, alcohols, tetrahydrofuran, acetonitrile, 1,4-dioxane, n-hexane, and cyclohexane.

10. The method of claim 8, wherein a molar ratio of the hydrogen to the carbon dioxide is between 0.5:1 and 5:1.
